(19) United States
(12) Patent Application Publication
(10) Pub. No.: US 2017/0196906 A1
(43) Pub. Date: Jul. 13, 2017

(54) NOBLE GAS TREATMENT OF ALZHEIMER'S DISEASE AND TAUPATHIES

(71) Applicant: NOBLIS THERAPEUTICS, INC., Portland, OR (US)

(72) Inventors: Vlad Bogin, Portland, OR (US); Thomas Ichim, San Diego, CA (US)

(21) Appl. No.: 15/402,177

(22) Filed: Jan. 9, 2017

Related U.S. Application Data
(60) Provisional application No. 62/276,753, filed on Jan. 8, 2016.

(51) Int. Cl. A61K 33/00; A61K 9/00

(52) U.S. Cl. CPC A61K 33/00; A61K 9/007; A61K 9/0043

(57) ABSTRACT

The invention discloses means of utilizing Noble Gases for inhibition of pathogenic processes associated with Alzheimer's Disease progression, as well as reversal of Alzheimer's Disease. In one embodiment compositions containing xenon gas are administered alone or together with therapeutic interventions for treatment of Alzheimer's Disease. In another embodiment, taupathies such as chronic traumatic encephalopathy are treated by administration of xenon or Noble Gas containing mixtures.

### RELATED APPLICATIONS

This application claims priority to U.S. Provisional Application No. 62/276,753, filed Jan. 8, 2016, which is incorporated herein by reference in its entirety

### BACKGROUND OF THE INVENTION

Alzheimer's Disease (AD), the most common form of dementia in the elderly, is characterized by the loss of neurons and synapses in the brain. AD affects an estimated 15 million people worldwide and 40% of the population above 85 years. The disease is characterized by progressive loss of memory, speech and movement with a total incapacitation of the patient and eventually death. AD takes a terrible toll on those with the disease as well as their families, friends and caregivers. Symptoms of AD manifest slowly and the first symptom may only be mild forgetfulness. In this stage, individuals may forget recent events, activities, the names of familiar people or things and may not be able to solve simple math problems. As the disease progresses into moderate stages of AD, symptoms are more easily noticed and become serious enough to cause people with AD or their family members to seek medical help. Moderate-stage symptoms of AD include forgetting how to do simple tasks such as grooming, and problems develop with speaking, understanding, reading, or writing. Severe stage AD patients may become anxious or aggressive, may wander away from home and ultimately need total care.

At a molecular level, amyloid plaques and neurofibrillary tangles are evident in the brains of patients with AD, marked by the presence of insoluble deposits and amyloid-beta peptide and cellular material surrounding the neurons. An essential disease process involves cleavage of the Aβ peptide from the integral membrane amyloid precursor protein (APP) by β-secretase to yield a secreted fragment of APP (sAPPβ) and a C-terminal fragment of APP (CTFβ) which undergoes further cleavage by the γ-secretase complex to yield a smaller C-terminal fragment (CTFγ) and Aβ fragments with prevalence of Aβ42 and Aβ40. Accumulation of Aβ fragments lead to their oligomerization, which mediates significant neurological damage. Accordingly, therapeutic interventions have been directed toward preventing Aβ over-production and deposition, accelerating Aβ degradation using β- and γ-secretase inhibitors/modulators, tau aggregation blockers, and immunotherapy.

Tau is a microtubule-associated protein expressed in the central nervous system whose main physiological purpose is the stabilization of microtubules. Tau is generated from one gene which possesses 6 isoforms, after alternative splicing. Under pathological conditions, most noticeably in Alzheimer's Disease, the tau protein becomes hyperphosphorylated, resulting in a loss of tubulin binding and destabilization of microtubules followed by the aggregation and deposition of tau in pathogenic neurofibrillary tangles. These pathologies are referred to as neurodegenerative tauopathies—are part of a group of protein misfolding disorders which besides Alzheimer's disease (AD), include progressive supranuclear palsy, Pick's disease, corticabasal degeneration, FTDP-17 among others. More than 40 mutations in tau gene have been reported to be associated with hereditary frontotemporal dementia demonstrating that tau gene mutations are sufficient to trigger neurodegeneration. Studies in transgenic mice and cell culture indicate that in AD, tau pathology may be caused by a pathological cascade in which A.beta. lies upstream of tau. Immunotherapies targeting the beta-amyloid peptide in AD have produced encouraging results in animal models and shown promise in clinical trials. More recent autopsy data from a small number of subjects suggests that clearance of beta-amyloid plaques in patients with progressed AD may not be sufficient to halt cognitive deterioration, emphasizing the need for additional therapeutic strategies for AD. In the wake of the success of Abeta-based immunization therapy in transgenic animal models, the concept of active immunotherapy was expanded to the tau protein. Active vaccination of wild type mice using the tau protein was however found to induce the formation of neurofibrillary tangles, axonal damage and mononuclear infiltrates in the central nervous system, accompanied by neurologic deficits

Numerous pre-clinical and clinical studies have demonstrated that robust immune responses consisting of high levels of anti-Aβ antibodies are beneficial for preventing the accumulation of toxic forms of Aβ in the brain. Vaccination strategies are being aggressively pursued to achieve high titers of anti-Aβ antibodies, while avoiding the potential that exists for eliciting autoreactive T cell responses. Current treatments for AD include cholinesterase inhibitors (donepezil, rivastigmine, galantamine) for mild to moderate AD, and the NMDA receptor antagonist (memantine) for moderate to severe AD. However, these drugs do not address the pathogenic mechanisms of disease and provide only short-term benefits. Over the past decade, Aβ immunotherapy has been tested using both active and passive immunization approaches, and might evolve as an effective strategy for preventing the accumulation of toxic forms of Aβ.

No cure is currently available for AD. Today, medication therapy focuses on controlling the symptoms of AD and its various stages. For example, mild to moderate AD can involve treatment with cholinesterase inhibitors such as Cognex® (tacrine), Aricept® (donepezil), Exelon® (rivastigmine), or Razadyne® (galantamine). Whereas moderate to severe AD can be treated with Namenda® (memantine). These medications may help delay or prevent AD symptoms from becoming worse for a limited period of time. So early AD treatment is warranted. However, there is no clear evidence that these medications have any effect on the underlying progression of the disease.

Lower levels of AB in the plasma are associated with cognitive decline in AD patients, arising as a result of increased influx of circulating AB into the brain. Hence, circulating AB levels are considered as being predictive of cognitive function. What is more, AB in the blood should be considered a therapeutic target, whereby depleting the peripheral reserve of AB will draw AB out of the brain and the physiological gradient that moves the neurotoxic peptide out of the brain will should lead to improved cognitive function.

### BRIEF DESCRIPTION OF THE DRAWINGS

FIG. 1 is a bar graph showing the suppression of inducible TNF alpha production from monocytes by xenon.

FIG. 2 is a bar graph showing the suppression of inducible IL-12 production from dendritic cells by xenon.

### DESCRIPTION OF THE INVENTION

The current invention provides means and methods for prophylaxis and treatment of Alzheimer's disease, and neuropathies associated with accumulation of amyloid deposits or tau mediated pathologies through administration of Nobel Gases, with preferred embodiments including administration of xenon, or xenon/argon mixtures.

In one aspect of the invention, means are provided to remove pathological proteins through administration of antagonists of the pathologic activity, while concurrently administering Nobel Gases, with preference to xenon or xenon/argon mixtures.

In other aspects, the invention provides means of using antibodies that target liposomes carrying xenon, or xenon/argon mixtures to site of plaque accumulation. In one embodiment the invention provides removing a circulating factor that is associated with pathology by extracorporeal means while concurrently administering xenon or xenon/argon mixtures, said factor being a form of amyloid beta (Aβ) protein, said specific form being selected from a group comprising of: a) a precursor of Aβ; b) a truncated variant of Aβ; c) a peptide derived from β; d) a pryoglutamated peptide of Aβ; e) a cross-linked beta amyloid protein species (CLAPS); f) an oxidized amyloid beta protein; g) amyloid precursor protein (APP); h) Aβ(1-42); i) Aβ(1-40); j) a peptide generated by enzymatic cleavage of APP. In another aspect, the type of Aβ removed from circulation is selected from a monomeric, a fibrillar, or a microvesicle associated form. Additionally, one of skill in the art may utilize removal of a circulating factor such as: a) tau protein, b) hyperphosphorylated tau protein; c) peptides derived from tau protein; and d) precursors to tau protein. Inhibition of said soluble, or circulating factors may be accomplished through suppression of biological (pathological) activity through binding of a ligand or inactivator factor, said factors are selected from a group comprising of: a) an antibody; b) an aptemer; c) a small molecule; and d) a microbody.

In one aspect of the invention the suppression of pathological activity is accomplished by immunization against antigens found on circulating pathological factors, with the immunization being performed oin a manner that is associated with minimal, to absent brain hemorrhage or microvascular pathology, specifically, various adjuvants may be used to modulate the immune responses, said adjuvants may be selected from a group of adjuvants comprising of: a) a modified dendritic cells; b) a polymer; c) a cytokine; d) a molecular entity possessing ability to inhibit inflammatory responses; e) a cytokine gene; and f) a molecular capable of eliciting RNA interference at the level of antigen presentation. In one aspect, dendritic cells that are made immature are utilized, wherein said immature state is defined by possession of phagocytic activity, as well as by substantially lower expression as compared to activation of said dendritic cells after treatment with 1 ug/ml LPS, of molecules selected from the group comprising of: a) MHC II; b) CD80; c) CD86; and d) IL-12. In one embodiment immunization is performed subsequent to, or concurrently with, xenon, or xenon/argon mixtures in order to decrease risk of neuronal damage associated with immunization.

Various means of immunization include loading antigen into dendritic cells, by administration of said antigen in the form of a protein, a modified protein, and a protein that has been altered to contain said original antigen, together with immune modulators that are selected from a group which includes: a) a cytokine; b) a heat shock protein; c) a ligand of a toll like receptor; and d) an activator of a pattern recognition receptor. Responses stimulated by immunization are immune responses that are primarily antibody mediated but do not inhibit endothelial integrity. Specifically, immune responses that are not associated with activation of Th1 or Th17 are desired. More specifically, antibody responses that do not give rise to antibodies that are complement fixing or stimulatory of antibody dependent cellular cytotoxicity. In one aspect the invention provides means of preventing progression of Alzheimer's Disease, or inducing regression, through the steps of administering into a patient an antibody capable of binding circulating factors associated with said disease, followed by removing of antigen-antibody complexes through extracorporeal extraction.

In one aspect, the invention uses administration of intravenous immunoglobulin (IVIG) as a source of premade antibody that binds antigens associated with Alzheimer's progression together with xenon, or xenon/argon mixtures. Said antigen-antibody complexes may be removed by the body naturally, or may be removed by means of peritoneal dialysis or extracorporeal dialysis means. In the aspect of the invention associated with peritoneal dialysis binding, patients are treated with microbeads or polymers, that are antibody conjugated microparticles being allowed to reside in said peritoneal cavity for a time-frame sufficient to allow extraction of said circulating ligand, followed by removal of said antibody conjugated microparticle from said peritoneal cavity.

Various aspects of the invention include the modulation of immunity through systemic administration of factors that may be useful in the inhibition of pathological immunity subsequent to administration of the antigen in an immunogenic fashion. Specific examples including immunization followed by administration of cytokines that shift systemic immunity, these include factors such as interleukin-4, interleukin-10, interleukin-13, and interleukin-20. Other means of immune modulation include the administration of immature monocytes, which would support for not only inhibition of pathological effects of the antibody but also promote regeneration of injured neural tissue through local and systemically secretion of growth factors. The invention provides methods of preventing and/or treating Alzheimer's disease and related neuropathies through selective inactivation of circulating neurotoxic/neuroinhibitory factors and precursors thereof. In one specific embodiment inactivation is preformed through administration of agents capable of suppressing biological activity of said circulating neurotoxic/neuroinhibitory factors and precursors thereof. Said agents are inhibitors of aggregation of amyloid subunits, as well as promoters of deaggregation of polymerized subunits. Specific means of inhibition include stimulation of antibody production towards known circulating factors such as forms of amyloid beta, including precursors of Aβ, truncated variants of Aβ, peptides derived from β, pryoglutamated peptides of Aβ, cross-linked beta amyloid protein species, oxidized forms of amyloid beta, amyloid precursor protein (APP), Aβ(1-42), Aβ(1-40), peptide generated by enzymatic cleavage of APP, monomeric, fibrillar, or microvesicle associated form of amyloid beta, tau protein, hyperphosphorylated tau protein; peptides derived from tau protein; and precursors to tau protein. Said antibodies may be administered passively after production in an in vitro, in vivo, or ex vivo system, may be monoclonal, polyclonal, or sera post immunization, may be fractionated or non-fractionated intravenous immunoglobulin, or may be immunoglobulins that have been generated as a result of immunization of the host with antigen. In the current invention antibodies are administered in a manner preventing significant complement deposition, microvascular leakage or hemorrahage, and/or with the concurrent inhibition of T cell type 1 or type 17 immunity.

In another embodiment, the invention provides means of suppressing accumulation of neurotoxic/neuroinhibitory factors by the binding to inhibitory agents, subsequent to which complexes of neurotoxic/neuroinhibitory factors with said inhibitory agents are removed. Said removal may be performed in a preferred embodiment by extracorporeal or peritoneal dialysis means.

In one embodiment, systems are disclosed that are useful for the reduction of plasma levels of antibodies bound to neurotoxic/neuroinhibitory factors, as well as reduction of plasma levels of said neurotoxic/neuroinhibitory factors without immunization or stimulation of antibody. Said neurotoxic/neuroinhibitory factors include alpha synuclein and peptides thereof, as well as a form of amyloid beta (Aβ) protein, said specific form being selected from a group comprising of: a) a precursor of Aβ; b) a truncated variant of Aβ; c) a peptide derived from β; d) a pryoglutamated peptide of Aβ; e) a cross-linked beta amyloid protein species (CLAPS); f) an oxidized amyloid beta protein; g) amyloid precursor protein (APP); h) Aβ(1-42); i) Aβ(1-40); j) a peptide generated by enzymatic cleavage of APP. In another aspect, the type of Aβ removed from circulation is selected from a monomeric, a fibrillar, or a microvesicle associated form. Additionally, one of skill in the art may utilize removal of a circulating factor such as: a) tau protein, b) hyperphosphorylated tau protein; c) peptides derived from tau protein; and d) precursors to tau protein. Said systems useful for reduction of said neurotoxic/neuroinhibitory factors include a medical apparatus or device such as a plasmapheresis system for removal of the blood from a patient, together with a means for separating the blood into plasma and cellular elements such as the red and white cells, such as a filter or a centrifuge, to which a means containing immobilized binding partners for said neurotoxic/neuroinhibitory factors which are bound to a column or a filter, together with a means for return of the plasma and separated and treated plasma to the patient, which usually consists of a tubing set. Said immobilized binding partners may be an antibody, a plurality of antibodies, an aptamer, a protein, a peptide, or a small molecule. Contemplated within the invention are systems, including plasmapheresis systems in which particulate elements of the blood are removed before extraction of neurotoxic/neuroinhibitory factors by affinity means, with one of skill in the art understanding that numerous systems exist for separating blood into the cellular components and plasma. Examples of such systems is the B. Braun Diapact CCRT plasma exchange/plasma profusion controller with plasma profusion tubing, as well as the Asahi and Kurray system, the Gambo Prisma System and the blood filtration controllers and the Fresenius Hemocare Apheresis system or the Exorim Immuoadsorption Systems. Filtration of the plasma and retention of the neurotoxic/neuroinhibitory factors, is performed within the context of the invention using a filter that is biocompatible, and suitable for contact with blood, without causing excessive activation of immune cells, platelets or clotting. Devices will typically be either parallel plate filters or capillary membrane filters. These can be adapted from devices currently in use for kidney dialysis. In one embodiment, the capillary membrane filters will typically have a surface area of between about 0.25 and 1 m(2) for use with children and between about 1 and 3 m(3) for use with adults. The parallel plate filters will typically have a surface area in the range from 0.1 and 2 cm.sup.2/ml of blood to be filtered. The filter membranes will typically be a biocompatible or inert thermoplastic such as urethane, polycarbonate, polytetrafluorethylene (Teflon.sup.R), polypropylene, ethylene polyvinyl alcohol or polysulfone. In some embodiments, It is often desirable to profuse proteins in the lower molecular weight fraction of the plasma, and avoid profusing large macromolecular proteins, such as fibrinogen, alpha 2 macroglobulin, and macroglobulins such as cryoglobulins, over the adsorber. Therefore membrane that possess molecular seiving discrimination in these molecular sizes are desirable. Such membranes ideally have a pore size typically of between 0.02 and 0.05 microns in a capillary membrane filter and of between 0.04 and 0.08 microns in a parallel plate filter. Polysulfone is preferred to ethylene vinyl acetate since it is more gentle towards the blood cells. The actual pore size that yields the desired cutoff is determined based on the flow geometry, shear forces, flow rate, and surface area. The effective cutoff for a capillary membrane filter with a pore size of 0.03 microns is 170,000 daltons, with a sieving coefficient of between 10 and 30%. The flow rate of plasma from these systems depends on the blood flow rate and the filter. At a flow rate of 300 ml blood/min (with a range of between 150 and 500 ml/min), the plasmapheresis systems typically yield a plasma flow rate of 100 ml filtrate (plasma)/min. The preferred range of flow rates is between 10 and 100 mL/min, with a more preferred range of between 50 and 100 ml. The filter may be bound with affinity agents or ligands capable of selectively, or semi-selectively binding neurotoxic/neuroinhibitory factors from circulations, said factors, described above, include alpha synuclein and peptides thereof, as well as a form of amyloid beta (Aβ) protein, said specific form being selected from a group comprising of: a) a precursor of Aβ; b) a truncated variant of Aβ; c) a peptide derived from β; d) a pryoglutamated peptide of Aβ; e) a cross-linked beta amyloid protein species (CLAPS); f) an oxidized amyloid beta protein; g) amyloid precursor protein (APP); h) Aβ(1-42); i) Aβ(1-40); j) a peptide generated by enzymatic cleavage of APP. In another aspect, the type of Aβ removed from circulation is selected from a monomeric, a fibrillar, or a microvesicle associated form. Additionally, one of skill in the art may utilize removal of a circulating factor such as: a) tau protein, b) hyperphosphorylated tau protein; c) peptides derived from tau protein; and d) precursors to tau protein. In another embodiment, a matrix of an adsorbant column is constructed in its geometry so as to couple the inhibitor binding ligands in microscopic pits on the surface of the bead so as to allow neurotoxic/neuroinhibitory factors to come in contact with the binding ligand (protein, aptamer, antibody or peptide) but prevent blood cells from coming in contact with the binding ligand. This system allows for the removal of the desired inhibitors from whole blood and makes the use of a filter unnecessary. In the practice of the invention, the patient will typically be connected to the blood processing device using an indwelling venous catheter and a standard intravenous tubing, with connections similar to those used for other extracorporeal blood treatment systems, so that blood can be removed from and returned to the patient. The tubing is connected to a blood pump that controls the flow rate so that in the preferred embodiment one blood volume (based on approximately 7% of the total body weight) is processed over a period of approximately 15-20 minutes. Other variants of this are possible based on clinical practice and minimal experimentation. After passing through the blood filter, the plasma filtrate is directed to the inhibitor removal column or filter, and then returned from these devices to the patient at either a single catheter site or a second site. Standard microprocessor controls can be used to regulate the blood flow, for example, by monitoring the volume of the blood products being removed, in combination with flow rate monitors and pump speed. In one preferred embodiment of the invention, plasmapheresis or hemodialysis technology will be utilized to deplete neurodegenerative peptides from the blood. In this embodiment, aptamers or antibodies specific for the target peptide will be immobilized onto plasmapheresis cartridges or functionally equivalent substrates, which can be fitted for existing hemofiltration infrastructure. Reduction in the circulating concentrations of neurodegenerative peptides would be monitored as a read-out for determining the suitability of the treatment regimen for reducing the concentrations of said factors in the blood.

In the scope of the invention, neurotoxic/neuroinhibitory factors can be removed by binding to the immobilized protein, antibody, an epitope or fragment thereof which selectively binds to the soluble neurotoxic/neuroinhibitory factor. As described here, the term “selectively binds” means that a molecule binds to one type of target molecule, but not substantially to other types of molecules. The term “specifically binds” is used interchangeably herein with “selectively binds”. As used herein, the term “binding partner” is intended to include any molecule chosen for its ability to selectively bind to the neurotoxic/neuroinhibitory factor. The binding partner can be one which naturally binds the targeted neurotoxic/neuroinhibitory factor. Antibodies can be polyclonal, monoclonal, recombinant, synthetic or humanized. Antibody fragments or single chain antibodies may also be used that bind to the inhibitor to be removed. Polyclonal antibodies may be are preferred since these have a broader range of reactivity and it is not necessary to have human antibodies since the antibodies are immobilized, not administered to the patient. The ligands or binding partners of said neurotoxic/neuroinhibitory factors can be immobilized in a filter, in a column, or using other standard techniques for binding reactions to remove proteins from the blood or plasma of a patient, or administered directly to the patient in a suitable pharmaceutically acceptable carrier such as saline or microbeads. Antibodies can be obtained from various commercial sources such as Life Technologiess. These are preferably humanized for direct administration to a human, but may be of animal origin if immobilized in an extracorporeal device with minimal leaching. Antibodies may be monoclonal or polyclonal. The antibodies and device should be prepared aseptically so as not to contain endotoxin or other materials not acceptable for administration to a patient. In one embodiment, plasma is circulated through an inert polymeric matrix, such as SEPHAROSE™, sold by Amersham-Biosciences, Upsala, Sweden, within a medical grade polycarbonate housing approximately 325 ml in volume, supplied by Tacoma Plastics. These should be sterilizable or produced aseptically and be suitable for connection using standard apheresis tubing sets. Typical materials include acrylamide and agarose particles or beads. Other suitable matrices are available, and can be formed of acrylamide or other inert polymeric material to which antibody can be bound. Standard techniques for coupling of antibodies to the gel material are used. In another embodiment, the binding partners are immobilized to filter membranes or capillary dialysis tubing, where the plasma passes adjacent to, or through, the membranes to which the binding partners are bound. Suitable filters include those discussed above with respect to separation of blood components. These may be the same filters, having immobilized binding partners bound thereto, or may be arranged in sequence, so that the initial filter separates the blood components and the subsequent filter removes the inhibitors. In another embodiment, the immobilized binding partners are bound to particles that are exposed to the blood or plasma within a mesh or reactor having retaining means.

In one embodiment the invention provides treatment of Alzheimer's using selective, or semi-selective delivery of xenon, or xenon/argon combinations to central nervous system tissue, particularly areas affected by Alzheimers such as hippocampal structures. Selective or semi-selective delivery can be accomplished utilizing xenon, xenon/argon containing liposomes. In one embodiment liposomes release their payload by attaching to a substrate, such attachment can be mediated by utilization of immunoliposomes that possess antibodies selective to the substrate desired. In situations where direct delivery of xenon, xenon/argon compositions is desired, selectivity is achieved by utilizing immunoliposome techniques as described in the following works which are incorporated by reference [1, 2]. In one embodiment liposomes are made loaded with noble gas mixtures and selectively dissociated using ultrasound or other means of energy transfer in order to release gaseous components.

In one embodiment, said liposomes are nanobubbles with a size less than 1 μm are created from lipid-encapsulated nanoparticles. Specifically, in manufacture dispersed phospholipid molecules in the prefabricated free nanobubbles solution are assembled to form controllable stable lipid encapsulation gas-filled ultrasound-sensitive liposome (GU-Liposome). In one embodiment GU-Liposomes have mean diameter of 194.4±6.6 nm and zeta potential of −25.2±1.9 mV with layer by layer self-assembled lipid structure. Production of this type of liposome is described in the following paper, which is incorporated by reference [3].

Liposomes may be made from L-alpha phosphatidylcholine Type XIII-E (PC), 3-(2-pyridyldithiolpropionic acid N-hydroxysuccinimide ester (SPDP) and cholesterol (Sigma Chemical Co., St. Louis, Mo.); maleimido-4 (p-phenylbutyrate)-phosphatidylethanolamine (MPB-PE) (Avanti Polar Lipids, Alabaster, Ala.); 1,2-dipalmitoyl-sn-glycero-3-phosphoglycerol (DPPG) (Fine Chemicals, Liestal, Switzerland); dithiothreitol (DTT) (Fisher Scientific, Itasca, Ill.); rabbit antihuman fibrinogen (American Diagnostica, Greenwich, Conn.), and intercellular adhesion molecule antibodies. In one embodiment Maleimido-4 (p-phenylbutyrate)-phosphatidylethanolamine, a maleimide-containing phospholipid, is included in the composition of the liposomes for conjugation to the antibodies. Suitable antibodies include anti-Tau antibodies, anti-phosphorylated tau antibodies, or anti-beta amyloid antibodies in situations where it is desirable to selectively target the liposomes to plaques. The maleimide group of MPB-PE reacts with thiol groups, forming a stable thioether bridge. A 60:8:2:30 molar mixture of PC, MPB-PE, DPPG and cholesterol is dissolved in chloroform. The solvent is then evaporated by a rotary evaporator (Labconco, Kansas City, Mo.), rotated at 120 rpm and immersed in a thermostatted water bath, with the temperature set at 50° C. under argon. The resulting lipid film is then placed in a dessicator under vacuum for 2 days for complete drying. The dry lipid film is then rehydrated with deionized water to give a concentration of 10 mg lipid/1 ml water. After removing the film from the walls of the flask, the dispersion is sonicated in a water bath sonicator (Fisher Scientific, Itasca, Ill.) until the mean size of the liposomes was approximately 500 nm. Liposome size is determined by quasielastic light scatter. Insertion of Nobel gases, such as xenon may be performed during this step. D-mannitol (0.2 mol/L) is added to the liposome suspension in a 1:1 vol/vol ratio, and this suspension is frozen overnight at −70° C. The frozen samples are then placed in a lyophilizer (Labconco, Kansas City, Mo.) for 2 days. The dried lipids are resuspended with 0.1 mol/L phosphate buffer, pH 7.4, to give a concentration of 10 mg lipid/1 ml buffer. After hand shaking and brief vortexing, the size of the resulting liposomes is again determined. The mean size of the liposomes is meant to remain under 1 μm.

For conjugation to antibodies that bind neurons, or specifically plaques associated with neurons an antibody solution is prepared by dissolving the protein in 0.1 mol/L phosphate buffer (pH 7.4) at a concentration of 5 mg ml⁻¹. A 20-μmol/ml⁻¹ solution of SPDP in ethanol. The SPDP solution is added to the stirred protein solution to give a molar ratio of SPDP to protein of 15:1. The mixture reacts for 30 min at room temperature. After reaction, the protein is separated from the reactants by gel chromatography. The column is then packed with Sephadex G-50 and equilibrated with 0.05 mol/L sodium citrate, 0.05 mol/L sodium phosphate and 0.05 mol/L sodium chloride at pH 7.0. Detection of protein in column elutions was determined by absorbance at 280 nm. The pyridyl dithio-protein solution is then titrated in citrate-phosphate buffer to pH 5.5 by addition of 1 mol/L HCl. A solution of 2.5 mol/L DTT in 0.2 mol/L acetate buffer, pH 5.5, was made. Ten microliters of DTT solution is added for every milliliter of protein solution. This mixture is allowed to stand for 30 min. The protein is then separated from the DTT by chromatography on a Sephadex G-50 column equilibrated with citrate-phosphate buffer at pH 7.0. Detection of protein in the elution is determined by absorbance at 280 nm. Argon was bubbled through all buffers to remove oxygen and where desired, addition of Nobel gas, in one preferred embodiment Xenon is used together with argon or in alone. The protein portion is then mixed with the MPB-PE-containing liposomes and allowed to react overnight under argon. Unbound protein is separated from the liposomes on a Sepharose 4B column.

For assessment of size after the product is generated, quasielastic light scattering measurements are performed using a Nicomp model 270 submicron particle sizer (Pacific Scientific, Menlo Park, Calif.) equipped with a 5-mW helium-neon laser at an exciting wavelength of 632.8 nm and with a 64-channel autocorrelation function, a temperature-controlled scattering cell holder and an ADM 11 video display terminal computer (Lear Siegler Inc., Anaheim, Calif.) for analyzing the fluctuations in scattered light intensity generated by the diffusion of particles in solution. The mean hydrodynamic particle diameter, dₕ, is then obtained from the Stokes-Einstein relation using the measured diffusion coefficient obtained from the fit.

### EXAMPLES OF THE INVENTION

### Example 1

### Suppression of Alzheimer's Peptide Induced Monocyte TNF-alpha Production by Xenon Gas

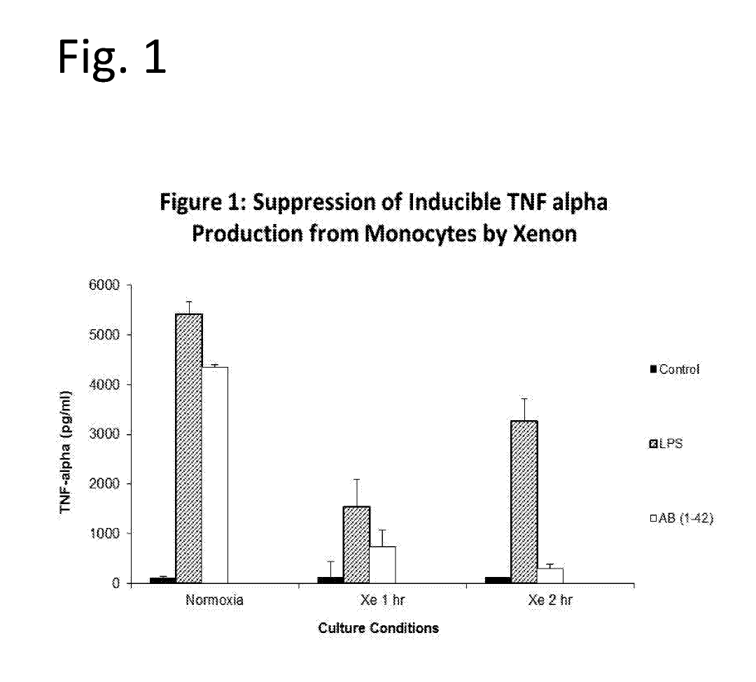

10 ml of heparinized blood was extracted from healthy volunteers and peripheral blood mononuclear cells were isolated by centrifugation over a ficoll gradient. Cells were subsequently washed in 2× volume phosphate buffered saline, and resuspended in RPMI media supplemented with 10% fetal calf serum. Cells were incubated at a concentration of 10 million cells per ml in 6-well plates in a volume of 2 ml per well. Cells were cultured for 24 hours at 37 Celsius in a fully humidified atmosphere with 5% CO2, subsequent to which non-adherent cells were removed by washing with PBS. Adherent cells were subsequently divided into 2 plates, one plate incubated at normoxic tissue culture, the second plate cultured for 1 hour in 30% xenon by volume, and the third tissue cultured for 2 hours in 30% volume. Cells were treated with control media, 1 ug/ml LPS and 10 μM of amyloid beta peptide Aβ (1-42) for 24 h. Supernatant was collected and assessed for TNF-alpha production by ELISA. As seen in FIG. 1, suppression of induced TNF-alpha production was observed in response incubation with xenon gas.

### Example 2

### Suppression of Alzheimer's Peptide Induced Dendritic Cell IL-12 Production by Xenon Gas

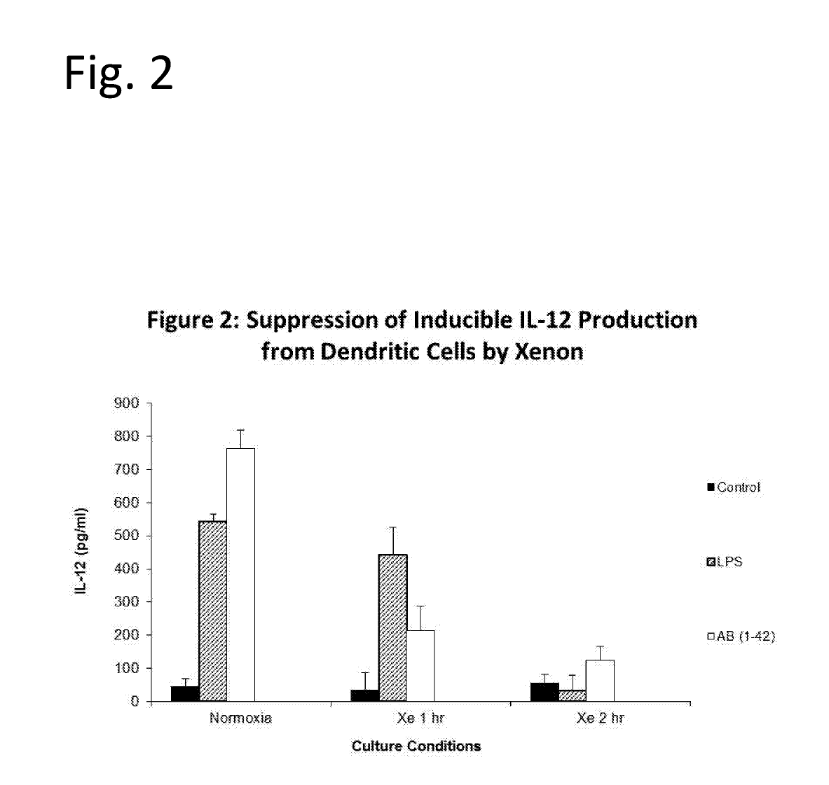

Monocytes from adherent PBMC were isolated as described in Example 1. Dendritic cells were generated by 7 day culture in IL-4 (10 ng/ml) and GM-CSF (20 ng/ml). Media was changed every two days. On day 7, purification of CD1c dendritic cells was performed and cells were cultured in control media, 1 ug/ml LPS and 10 μM of amyloid beta peptide Aβ (1-42) for 24 h. Supernatant was collected and assessed for IL-12 production by ELISA. As seen in FIG. 2, suppression of induced IL-12 production was observed in response incubation with xenon gas.

### REFERENCES

1. Lin, Q., et al., Brain tumor-targeted delivery and therapy by focused ultrasound introduced doxorubicin-loaded cationic liposomes. Cancer Chemother Pharmacol, 2015.

2. Yu, F. T., et al., Low Intensity Ultrasound Mediated Liposomal Doxorubicin Delivery Using Polymer Microbubbles. Mol Pharm, 2015.

3. Tian, J., et al., A Novel Approach to Making the Gas-Filled Liposome Real: Based on the Interaction of Lipid with Free Nanobubble within the Solution. ACS Appl Mater Interfaces, 2015. 7 (48): p. 26579-84.

1. A method of treating Alzheimer's disease or progression to Alzheimer's disease comprising the steps of: a) identifying a patient with Alzheimer's disease or pre-Alzheimer's disease state; b) administering a noble gas containing composition at a sufficient concentration and frequency to reduce pathogenic mechanisms associated with Alzheimer's disease pathology, or progression to Alzheimer's disease; and c) adjusting said concentration of frequency of said noble gas containing composition based on said responses of said patient in terms of modulation of mechanisms associated with Alzheimer's disease pathology or progression to Alzheimer's disease.

2. The method of claim 1, wherein said Alzheimer's disease is characterized by a group of biological activities consisting of: a) excessive excitotoxicity as compared to an age-matched individual; b) formation of beta amyloid plaques; c) increased ratio of pERK2 over pERK1 in neurons; d) cognitive impairment; e) excess phosphorylated tau in neurons as compared to healthy age-matched controls; f) neuroinflammation and g) increase neuronal apoptosis.

3. The method of claim 1, wherein said pre-Alzheimer's disease state is characterized by a group of biological activities consisting of: a) mild cognitive impairment; b) detection of phosphorylated tau in systemically circulating microvesicles; c) detection of phosphorylated tau in systemically circulating exosomes; and d) excessive excitotoxicity as compared to an age-matched individual;

4. The method of claim 1, wherein said pre-Alzheimer's disease and Alzheimer's disease are associated with microglial activation, said activation higher than age-matched healthy controls.

5. The method of claim 4, wherein said microglial activation consists of properties selected from a group comprising of: a) TNF-alpha production; b) IL-12 production; c) IL-1 beta production; d) IL-6 production; e) IL-33 production; and f) BDNF production.

6. The method of claim 4, wherein said microglial activation consists of reduced production as compared to unactivated microglial cells of cytokines selected from a group comprising of: a) IL-10; b) IL-20; and c) TGF-beta.

7. The method of claim 1, wherein said noble gas containing composition contains xenon and/or a xenon donor and at least one gaseous component selected from the group consisting of: a) oxygen, b) nitrogen, and c) argon.

8. The method of claim 7, wherein said noble gas containing composition comprises xenon gas.

9. The method of any of claim 8, wherein the xenon gas is administered at a concentration of 10% to 35% by volume in 21% by volume oxygen gas and a balance of nitrogen gas.

10. The method of claim 2, wherein said neuroinflammation is identified by enhanced plasma levels of cytokines associated with inflammation.

11. The method of claim 2, wherein said enhanced neuroinflammation is identified by reduced plasma levels of cytokines associated with reduction in inflammation.

12. The method of claim 1, wherein said noble gas containing mixture is comprised of a gas mixture containing oxygen and a proportion by volume of 20 to 70% of xenon.

13. The method of claim 12, wherein said proportion of xenon is between 22 and 60% by volume to oxygen.

14. The method of claim 12, wherein said proportion of xenon is between 25 and 60% by volume to oxygen.

15. The method of claim 1, wherein said noble gas containing mixture consists only of a) oxygen and xenon or b) air and xenon.

16. The method of claim 1, wherein said noble gas containing mixture also contains nitrogen, helium, Nitric Oxide, krypton, argon or neon.

17. The method of claim 1, wherein said noble gas containing mixture contains a proportion by volume of oxygen of between 15 and 25%.

18. The method of claim 1, wherein said noble gas containing mixture is supplied for inhalation from a pressurized container at a pressure greater than 2 bar.

19. The method of claim 1, wherein said noble gas containing mixture is administered intranasally.

20. A method of inhibiting production of TNF-alpha from a monocyte that has been stimulated with amyloid beta peptides.

21. The method of claim 20, wherein said amyloid beta peptide is AB (1-42).

22. A method of treating neuroinflammation mediated by dendritic cells through administration of a therapeutic dose of xenon.

23. The method of claim 22, wherein said neuroinflammation is mediated by production of IL-12 by dendritic cells.

24. The method of claim 23, wherein said neuroinflammation is stimulated by amyloid beta peptides.

25. The method of claim 24, wherein said amyloid beta peptide is AB (1-42).
